(19) United States
(12) Patent Application Publication
(10) Pub. No.: US 2008/0305187 A1
(43) Pub. Date: Dec. 11, 2008

(54) Use of Parathyroid Hormone for Treating Periodontal Disease and for Other Dental Applications

(76) Inventor: John Henry Krege, Carmel, IN (US)

Correspondence Address: ELI LILLY & COMPANY, PATENT DIVISION, P.O. BOX 6288, INDIANAPOLIS, IN, 46206-6288, US

(21) Appl. No.: 12/094,309

(22) PCT Filed: Dec. 19, 2006
(86) PCT No.: PCT/US06/62280

§ 371 Date: May 20, 2008

(30) Foreign Application Priority Data
Dec. 20, 2005 (US) 60/752130

(51) Int. Cl. A61K 38/29; A61K 33/06; A61P 29/00; A61K 38/23; A61P 19/00

(52) U.S. Cl. 424/682; 514/12

(57) ABSTRACT

Method for treating or preventing periodontal disease associated with mild to severe alveolar bone loss, and for improving the prognosis of dental implant procedures by administering human parathyroid hormone to a patient in need thereof.

### BACKGROUND OF THE INVENTION

The present invention relates generally to the field of dental disease and bone loss. More particularly the invention relates to therapeutic use of parathyroid hormone in treating conditions associated with periodontal disease.

### BRIEF SUMMARY OF THE INVENTION

Periodontal disease in the advanced stages is characterized by resorption of alveolar bone, loss of soft-tissue attachment, excessive tooth mobility, and, sometimes, loss of one or more teeth. It is the leading cause of tooth loss in adults, affecting 50 million people in the United States alone. Only about 15% of individuals afflicted with periodontal disease are adequately treated. Periodontal disease has also been associated with an increased risk of heart disease and stroke, as well as pre-term low birthweight infants, and therefore is a significant health concern.

Periodontal disease initiation and progression are dependent upon the presence of specific types of bacteria in the dental plaque as well as the host response (inflammatory and immune) to these bacteria. Recognized bacterial species associated with periodontal disease include Actinobacillus actinomycetemcomnitans and Porphyromonas gingivalis, both of which secrete a toxin that damages the surrounding tissues. The host response elicited by dental plaque accumulation can also cause local tissue damage. As a result of this bacterial- and host-mediated damage, the periodontal ligament is destroyed and alveolar bone loss ensues. Clinical signs of destruction include increased attachment level and pocket probing depth measurements around the tooth, and radiographic evidence of local alveolar bone loss.

Lost alveolar bone associated with periodontal disease not only increases the risk of loss of one or more teeth, it also may preclude or subvert ancillary dental replacement therapies including dental implants.

Current management of periodontal disease involves dental prophylaxis, scaling and root planning, and, in severe cases, periodontal surgery may be necessary. Therapeutic agents for treating the disease include antiseptic agents and/or local/systemic antibiotics or microbial agents to control plaque-causing bacteria, and anti-inflammatory agents that reduce the host inflammatory response. While these methods can be effective in controlling or slowing the progression of periodontal disease, limited alveolar bone is restored.

Alveolar bone infill has been achieved with various degrees of success using surgically implanted materials and topical agents, including bone grafts and bone morphogenetic proteins (BMPs). Guided tissue regeneration involves surgical placement of a membrane barrier between the bony defect and the overlying soft tissue in an attempt to stimulate the formation of a new periodontal attachment as well as bone formation.

The rate of alveolar bone loss with periodontal disease is likely influenced by both local and systemic host factors. Whether systemic bone loss as a result of osteopenia or osteoporosis also has detrimental effects on alveolar bone has not been established. Systemic bone loss associated with osteoporosis can be treated with parathyroid hormone (PTH), which increases BMD and reduces the risk of vertebral and non-vertebral bone fracture. (R. M. Neer et. al., NEJM, 19, 1434-1441, 2001). Since there remains a need for a treatment of periodontal disease that will restore lost alveolar bone, the present invention relates to administering parathyroid hormone (PTH) alone or in combination with one or more other agents to humans or animals to restore lost alveolar bone associated with periodontal disease and prevent further bone loss. As a consequence of alveolar bone infill, pocket probing depth and clinical attachment level is expected to improve.

An objective of the present invention is to reverse alveolar bone loss including bone loss associated with periodontal disease by the administration of an effective amount of parathyroid hormone to a patient in need thereof.

Another objective of the invention is to induce new alveolar bone formation in a patient in need thereof by the administration of an effective amount of parathyroid hormone.

Still another objective of the invention is to increase alveolar bone height by the administration of an effective amount of parathyroid hormone to a patient in need thereof.

Yet another objective of the invention is to increase alveolar bone width by the administration of an effective amount of parathyroid hormone to a patient in need thereof.

Another objective of the invention is to increase alveolar bone density by the administration of an effective amount of parathyroid hormone to a patient in need thereof.

Another objective is to decrease pocket probing depth and/or reduce clinical attachment level by the administration of an effective amount of parathyroid hormone to a patient in need thereof.

Still another objective of the present invention is to reduce tooth mobility, by the administration of an effective amount of parathyroid hormone to a patient in need thereof.

A further objective of the present invention is to restore alveolar bone that has been lost at the alveolar ridge following tooth extraction by administering a suitable dose of parathyroid hormone.

Another objective of the present invention is to reduce the pocket probing depth in a patient with periodontal disease by administering a suitable dose of parathyroid hormone.

Another objective of the invention is to induce new alveolar bone formation in a patient who has undergone or is about to undergo a dental implant procedure, with or without ancillary bone grafting procedure, wherein said new alveolar bone formation provides additional support for an implant device, by administering a suitable dose of parathyroid hormone.

Another objective of the invention is to induce alveolar ridge bone formation in a patient wearing dentures by administering a suitable dose of parathyroid hormone.

Still another objective of the invention is to provide stability to teeth of poor prognosis due to alveolar bone loss associated with periodontal disease, thereby reducing the risk of further tooth loss.

These and other objectives of the invention will be apparent to those skilled in the art and from the following detailed description of a preferred embodiment of the invention.

### DETAILED DESCRIPTION OF THE INVENTION

The term “calculus” means a hard deposit of mineralized plaque (also referred to as “tartar”).

The term “osseointegration” generally refers to bone growth or bonding onto the surface of an implant, thereby locking or stabilizing the implant in place.

The term “pharmaceutically-acceptable composition” means a formulation comprising PTH that is suitable for pharmaceutical use in humans. A composition may be in any form, such as a solution, suspension, freeze-dried pellet, oil, cream, tablet, capsule or ointment. Preferably, the composition is in a sterile, ready to use solution form, for example, a ready to inject form. A composition of PTH may be available for administration in any of a variety of containers, including a vial, bottle, jar, cartridge or pen injector device.

The term “pharmaceutically-acceptable carrier” means a solid or liquid chemical that is essentially inactive pharmacologically but is compatible with and suitable for pharmaceutical use in humans. Examples of pharmaceutically acceptable carriers include water, mannitol and lactose. A preferred carrier is water.

The term “parathyroid hormone” means full-length human parathyroid hormone (“PTH”) 1-84 as well as biologically active fragments thereof including PTH(1-9), PTH(1-11), PTH(1-14), PTH(1-20), PTH(1-28), PTH(1-31), PTH(1-34), PTH(1-35), PTH(1-37), PTH(1-38), PTH(1-41) and PTH(1-64). The term also includes analogs of full-length PTH or fragments thereof including amino acid substitutions and derivatives. Said analogs may optionally comprise one or more polyethylene glycol (PEG) or Fc fragments.

The term “pocket probing depth” (PPD) refers to the distance, measured in millimeters, from the base of the periodontal pocket to the gingival (i.e. glum) margin.

The term “clinical attachment level” (CAL) refers to the distance, measured in millimeters, from the cemento-enamel junction (i.e. crown margin) to the base of the pocket. Both PPD and CAL measurements are made with a periodontal probe at various sites around each tooth, for example the mesiobuccal or midbuccal sites. They provide a measure of the severity of periodontal disease.

The term “periodontal disease” as used herein is meant to encompass a spectrum of clinical conditions including 1) plaque-induced and non-plaque-induced gingival diseases, 2) chronic periodontitis, classified as slight (1-2 mm CAL), moderate (3-4 mm CAL), or severe (>5 mm CAL) of generalized or localized involvement, 3) aggressive periodontitis, classified as slight (1-2 mm CAL), moderate (3-4 mm CAL), or severe (>5 mm CAL) of generalized or localized involvement, 4) periodontitis as a manifestation of systemic diseases associated with hematologic disorders, genetic disorders, or not otherwise specified, 5) necrotizing periodontal diseases including necrotizing ulcerative gingivitis and necrotizing ulcerative periodontitis, 6) abcesses of the periodontium including gingival, periodontal and pericoronal abscesses, 7) periodontitis associated with endodontic lesions, and 8) developmental or acquired deformities and conditions, for example, localized tooth-related factors that modify or predispose to plaque-induced gingival diseases or periodontitis, mucogingival deformities and conditions around teeth, and conditions on edentulous ridges and occlusal trauma. All of these conditions may be localized to one or a few specific teeth, or more generalized (i.e. >30% of sites are involved). The severity of disease is usually based on the clinical attachment levels measured from the cementoenamel junction or crown margin, and may be considered mild (1-2 mm), moderate (3-4 mm), or severe (>5 mm). Severity of disease may also be based on pocket probing depths, with mild disease characterized by a pocket probing depth of about 4-5 mm, moderate disease characterized by a pocket probing depth of about 6-7 mm, and severe disease characterized by pocket probing depths about ≧8 mm.

### Alvcolar Bone Loss

Alveolar bone loss is central to the pathogenesis of progressive periodontal disease. In some cases oral bone loss may occur in conjunction with systemic bone loss typically associated with osteopenia and osteoporosis. It is unclear whether there is a causative relationship between systemic bone loss and periodontal disease, or whether systemic bone density measurements are a reliable predictor of mandibular bone density and/or risk of oral bone loss. Alveolar bone loss is influenced by multiple risk factors including host-mediated immune responses to elevated cytokine levels associated with periodontal disease. Osteoclast activity associated with periodontal disease is augmented in the host-mediated response.

Restoration of Lost Alveolar Bone Associated with Periodontal Disease

The method of the present invention can be applied to animals or humans exhibiting vertical and/or horizontal alveolar bone loss including bone loss associated with periodontal disease, with or without systemic bone loss associated with osteopenia or osteoporosis or other systemic bone loss disease. Patients having mild to severe alveolar bone loss can be treated by the method of the present invention to retard and/or prevent further loss of oral bone and to regenerate lost bone. The method of the invention increases bone density, bone mass, and bone strength of alveolar bone that has been weakened or destroyed, for example, during progression of periodontal or other degenerative disease. Application of the method of the invention lowers the risk of additional alveolar bone loss and stabilizes any remaining teeth, thereby reducing the risk of further tooth loss.

The method of the invention can be applied, for example, to treat adult periodontitis in men and women, and early-onset periodontitis affecting young adults, adolescents and children, including prepubertal periodontitis. The method is also suitable for treating localized or generalized periodontitis as well as rapidly progressive periodontitis. Juvenile forms of the disease are distinguished from adult onset forms by the age of onset, and often by the rate of disease progression, and the kinds of bacteria associated with the disease. Certain patients may be at higher risk of periodontal disease including post-menopausal women, diabetics, smokers, and steroid users.

The method is also applicable to other forms of periodontal disease including disease associated with metabolic disorders such as diabetes mellitus, and hormonal alterations associated with, for example, menopause. Use of certain drugs such as anticonvulsants, calcium channel blockers, and cyclosporine, may also elevate the risk of gingival hyperplasia or periodontal disease, as does certain hematologic disorders including immune system disorders caused by, for example, HIV.

Adult periodontitis is characterized by loss of clinical attachment due to destruction of the periodontal ligament, and by loss of adjacent supporting bone tissue in an otherwise healthy adult. Moderate adult periodontitis is characterized by loss of up to about 30% of supporting periodontal tissues, whereas advanced or severe adult disease exhibits loss of more than 30% of periodontal tissue which can be detected, for example, by radiographic analysis. Patients may experience periodontal destruction in a localized fashion, involving one area of a tooth or teeth, or the disease may be more general, involving multiple teeth or the entire dentition. Juvenile periodontitis may also present in a localized or general form.

The method of the present invention is suitable for treating various presentations of periodontal disease and to effect one or more clinical benefits including reduction in the rate of alveolar bone loss, reversal of alveolar bone loss, inducement of alveolar bone formation, increase in oral bone mineral density (BMD), reduced pocket probing depth and clinical attachment level, stabilization of teeth, reduction in the risk of tooth loss and/or stabilization of dental implants.

The method of the present invention can also be applied to patients having one or more independent or associated systemic bone loss diseases such as, for example, osteoporosis and/or osteopenia, including drug-induced osteoporosis, e.g. steroid-induced osteoporosis.

The method of the present invention can also be applied to individuals who may have lost one or more teeth, whether through caries, trauma or periodontal disease, and who also may have lost surrounding alveolar bone. Bone loss in these areas may continue over time. In some individuals, particularly in older individuals who may have lost a number of teeth at a young age there may be difficulties in wearing dentures, due to limited remaining bone support for the denture. This leads to problems with function and denture retention. The method of the invention can be used to help restore the ridge in such cases and reduce the problems associated with wearing dentures where there is limited bone support.

Use with Dental Implants

Another aspect of the present invention involves administration of parathyroid hormone to increase alveolar bone mass in a patient who has, or is about to have, a dental implant procedure. The method is particularly useful in patients who have inadequate alveolar bone to support an implant. Parathyroid hormone may be administered to accelerate osseointegration of dental implants, thereby increasing the stability of dental implants.

Prospective implant patients are examined by a dentist, periodontist, or oral surgeon, and implant procedures carried out to replace a single tooth, several teeth, or an entire upper or lower arch. Parathyroid hormone may be administered before or after an implant procedure. Prospective implant patients having inadequate or insufficient alveolar bone mass or density for a successful implant procedure are administered PTH for a period of about 1 month to about 6 months, preferably about 1 month to about 3 months, prior to an implant procedure in order to build sufficient alveolar bone mass for the procedure.

Parathyroid hormone may also be administered after a dental implant procedure in order to accelerate osseointegration of an implant device, and/or to improve or increase the stability of an implant. Post-implantation administration of PTH may be for about 3 months to about 12 months; preferably about 3 months to about 9 months; most preferably, about 3 months to about 6 months following an implant procedure.

The invention also contemplates optionally administering PTH in conjunction with other treatments and/or procedures applied prior to, during, or after a dental implant procedure including, for example, bone grafts, such as autografts, allografts, xenografts and alloplastic grafts, and/or administration of other bone active agents such as, for example, bone growth factors, e.g. BMPs, anti-bone resorption agents, e.g. AMG-162, bisphosphonates, estrogen, SERMs, calcitonin, cholecalciferol, vitamin D, and calcium.

### Administering Parathyroid Hormone

Parathyroid hormone can be administered parenterally or non-parenterally by any suitable route, including, for example, nasal, oral, buccal, pulmonary, intravenous, subcutaneous or intramuscular route, preferably by subcutaneous injection. PTH may also be administered by local injection within the oral cavity, preferably at a site or socket at which bone loss has occurred.

Parathyroid hormone can be formulated as a dried powder for nasal or pulmonary administration, freeze-dried formulations for reconstitution and injection, or ready to administer injectable solution formulations. Preferably the formulation is a ready to administer solution formulation, for example, as disclosed in U.S. Pat. No. 6,770,623, incorporated herein by reference.

Stabilized solution formulations of the present invention may include a parenterally acceptable preservative. Suitable preservatives include, for example, cresols, benzyl alcohol, phenol, benzalkonium chloride, benzethonium chloride, chlorobutanol, phenylethyl alcohol, methyl paraben, propyl paraben, thimerosal and phenylmercuric nitrate and acetate. A preferred preservative is m-cresol or benzyl alcohol; most preferred is m-cresol. The amount of preservative employed may range from about 0.1 to about 2 wt-%, preferably about 0.3 to about 1.0 wt-% of the total solution.

Parathyroid hormone compositions can, if desired, be provided in a powder form containing not more than 2% water by weight, after freeze-drying of a sterile, aqueous hormone solution prepared by mixing sterilized forms of the selected parathyroid hormone, buffering agent or water, and a stabilizing agent as described above. Especially useful as a buffering agent when preparing lyophilized powders is a tartrate source. Particularly useful stabilizing agents include glycine, sucrose, trehalose, marmitol, leucine, DPPC, and raffinosc.

Parathyroid hormone can also be formulated using any other suitable buffers and excipients to stabilize PTH. Pharmaceutical carriers and their formulations are described in Martin, “Remington's Pharmaceutical Sciences,” 15th Ed.; Mack Publishing Co., Easton (1975).

In one embodiment of the present invention PTH is administered concurrently or sequentially with one or more other agents, drugs, vitamins and/or nutritional supplements. Suitable agents according to this aspect of the invention include, inter alia, SERMS, raloxifene hydrochloride, calcitonin, bisphosphonates, growth factors, e.g. bone morphogenetic proteins such as BMP-2, estrogen or hormone replacement therapy, and calcium and/or vitamin D, the latter two agents either through dietary intake, or by dietary intake plus supplementation.

Preferably, PTH administered according to the present invention is human PTH(1-34), though other PTH fragments or fall-length PTH(1-84) hormone can also be used. Parathyroid hormone is administered at a dose of about 5 μg to about 500 μg per day, more preferably about 5 μg to about 250 μg per day; 5 μg to about 100 μg/(day, 10 μg to about 200 μg per day; 10 μg to about 100 μg per day; or about 10 μg to about 50 μg per day. Most preferably, PTH(1-34) is administered at a dose of about 5 μg to about 50 μg per day; alternatively 15 μg to about 50 μg per day; 20 μg to 40 μg per day; or 20 μg to about 30 μg per day. Alternatively, PTH(1-34) may be administered daily at a dosage of about 5, 10, 15, 20, 25, 30, 35, 40, 45, 50, 55, 60, 65, 70, 75, 80, 85, 90, 95, or 100 μg. Corresponding dosages of full length PTH or PTH fragments other than PTH(1-34) are based on molar equivalencies to PTH(1-34).

Parathyroid hormone can be administered regularly (e.g., once or more each day or week), intermittently (e.g., irregularly during a day or week), or cyclically (e.g., regularly for a period of days or weeks followed by a period without administration). Preferably, PTH is administered once daily for 1-7 days for a period ranging from one month up to 3 years. Alternatively PTH may be administered for any period from 1 month to 2 years. More preferably, PTH is administered once daily for a period of 3 months to 2 years; most preferably, PTH is administered once daily for a period of 3 months to 12 months.

When PTH is co-administered with one or more other bone active agents, such co-administration may be concurrent or sequential. Sequential treatment regimens include administration of bone-active agents other than PTH during a period or periods in which PTH is not administered. By way of example, PTH may be administered regularly or intermittently for 6 months followed by a period in which PTH is not administered but some other bone active agent is administered. Co-administration of a bone active agent(s) other than PTH may occur prior to or after a period in which PTH is administered, for about 1 month to 18 months; alternatively about 1 month to 12 months; or about 1 month to 6 months. Concurrent treatments include, for example, regimens in which PTH is administered together with one or more other bone active agents at the same time, including on the same days or alternating days within the same treatment period. Bone-active agents other than PTH may be co-administered with PTH according to this aspect of the invention at any suitable dosage known to the skilled artisan. For example, hormone replacement therapy (HRT) may be provided at dosages in the range of 0.5-1 mg of oral 17β-oestradiol, 25 μg of transdermal 17β-ocstradiol, or 0.3 mg of conjugated equine estrogens. Bisphosphonates such as alendronate, and risedronate may be administered at dosages in the range of 5-10 mg daily; calcitonin may be administered, for example, at 100-500 IU per day, and raloxifene at 20-200 mg per day; preferably 60-120 mg per day. These dosages are merely exemplary and other dosages known to the skilled artisan may be used.

One skilled in the art will recognize that the present invention may encompass numerous and varied treatment regimens, including dosage of PTH, route and site of administration, specific PTH or fragment thereof administered, formulation, treatment period, and, if appropriate, interval period between days that include PTH administration and days without PTH administration. Each treatment option may operate independently and may be adjusted as needed to achieve the desired therapeutic objective.

In one embodiment of the invention, a patient having mild periodontal disease and minimal alveolar bone loss receives once daily, once every other day, or once weekly subcutaneous administration of PTH for a period of one month to twelve months. Patients having moderate periodontal disease can be treated by once daily, once every other day, or once weekly administration of a suitable dose of PTH for a period of three months to twelve months; preferably six months to twelve months. Patients having severe periodontal disease can be treated by once daily, once every other day, or once weekly administration of a suitable dose of PTH for a period of three months to eighteen months; preferably six months to twelve months; most preferably nine months to twelve months.

While preferred embodiments of the invention have been shown and described, it will be apparent to those skilled in this art that various modifications may be made in these embodiments without departing from the spirit of the present invention.

### EXAMPLE 1

### Treatment of Patient with Periodontal Disease Prior to Dental Implant Therapy

A 65 year-old caucasian male presents with a history of adult periodontitis. The patient has experienced significant alveolar bone loss confirmed radiographically, and has a number of tooth sites with pocket probing depth measurements of about 7 mm. The patient has discussed with his dentist possible extraction of severely-affected teeth and their replacement with dental implants, but the prospects for success are diminished in view of the bone loss that has occurred. Standard periodontal care is provided, including scaling and root planning, and those teeth with poor prognosis are extracted. Prior to implant surgery, the patient is administered 20 μg of hPTH(1-34) once-daily by subcutaneous injection for 3 months prior to implant surgery. Following treatment with PTH, radiographic analysis reveals increased alveolar BMD, and reduced pocket probing depths around the remaining teeth. No complications occur post placement of the implants.

### EXAMPLE 2

### Treatment of Patient with Moderate to Severe Periodontal Disease

A 75-year old postmenopausal female without osteoporosis or any other systemic bone loss condition presents for tooth extraction. The patient has a long history of severe and chronic gum-bleeding associated with chronic gingivitis but is otherwise in good health. Clinical examination and radiographic analysis show significant alveolar bone loss, and pocket probing depth measurements, and a diagnosis of severe periodontal disease is made.

The patient receives standard periodontal treatment including scaling and root planning, with surgery if necessary, and self-injects 15 μg once per day of hPTH(1-34) subcutaneously for 12 months. Based on restricted dietary habits the patient is advised to take daily supplemental calcium and vitamin D. Pocket probing depth measurements are taken every three months for a period of 12 months. At the end of the 12-month period the patient exhibits reduced pocket probing depths and radiographic analysis reveals restoration of lost alveolar bone.

### EXAMPLE 3

### PTH(1-34) Administration Reduces Probe Depth in a Patient with Adult-Onset Periodontal Disease

A 40-year old male smoker presents with aggressive periodontal disease. The patient has already incurred several tooth extractions and there is evidence of advanced alveolar bone loss that threatens the remaining teeth. The patient receives intense periodontal treatment including scaling and root planing and begins subcutaneous injections of hPTH(1-34) once daily at a dose of 30 μg per day. Pocket depths are measured at regular intervals during a 12 month treatment period. After 12 months, the patient shows marked improvement, with fewer sites exhibiting deep pocketing.

### EXAMPLE 4

### PTH Administration to Repair Bony Defects Associated with Early Onset Periodontitis

A 25-year old woman presents with early-onset periodontitis, characterized by advanced bone loss including vertical interproximal defects and molar furcation involvement. She also exhibits increased tooth mobility and anterior incisor drifting. Along with intense periodontal treatment, including scaling and root planning, human PTH(1-34) is administered at a dose of 30 μg per day by subcutaneous injection along with debridement to repair the bony defects. After 6 months treatment the patient displays tooth stabilization.

### EXAMPLE 5

### Dental Implant Applications

A 55-year old woman presents with advanced oral bone loss as a result of periodontal disease and has already lost multiple teeth. She is keen to save her remaining teeth and replace those that have been lost with implants as soon as possible. She is treated with hPTH(1-34) at a dosage of 25 μg once daily by subcutaneous injection, along with debridement. Three months after the start of PTH(1-34) treatment the patient receives implants and continues to receive PTH for 3 additional months after the procedure. The implants are stabilized and functional within 3 months of placement. She retains her remaining teeth during 24 months of additional observation.

### EXAMPLE 6

### Treatment of Early-Onset Periodontal Disease

A 15-year old girl presents having already lost several teeth to aggressive juvenile periodontal disease. Radiographic analysis shows significant alveolar bone loss. She receives intense periodontal treatment and is subsequently treated for 3 months with PTH(1-34) given three days per week at a dose of 10 μg per day by subcutaneous injection, followed by 3 additional months with once daily injection at a dose of 20 μg per day. She exhibits improvement in her clinical attachment levels and bone infill around affected teeth. No more teeth are lost during follow up. Dental implants are used to successfully replace the teeth lost due to periodontal disease.

1. A method for the treatment of periodontal disease in a human patient comprising administration of human parathyroid hormone in a daily dose of 20 μg to 30 μg.

2. (canceled)

3. The method as in claim 1 wherein said treatment restores lost alveolar bone at one or more affected tooth sockets following tooth extraction.

4. The method as in claim 1 wherein said treatment reduces pocket probing depth.

5. The method as in claim 1 wherein said treatment increases alveolar BMD.

6. The method as in claim 1 wherein said periodontal disease is selected from the group consisting of juvenile periodontitis, adult periodontitis, necrotizing ulcerative gingivo-periodontitis, refractory periodontitis, early-onset periodontitis, aggressive periodontitis, and periodontitis associated with systemic disease.

7. (canceled)

8. The method as in claim 1 said treatment further comprising co-administering an agent selected from the group consisting of vitamin D, calcium, estrogen, hormone replacement therapy, bisphosphonate, calcitonin, and SERM.

9. The method as in claim 1 wherein said treatment with parathyroid hormone is for a period of one month to 24 months, and wherein at the end of said treatment period one or more other agents are administered, selected from the group consisting of vitamin D, calcium, estrogen, hormone replacement therapy, calcitonin, bisphosphonate, and SERM.

10. The method as in claim 1 wherein said parathyroid hormone is selected from the group consisting of PTH(1-28), PTH(1-31), PTH(1-34), PTH(1-37), PTH(1-38), PTH(1-41) and PTH(1-84).

11. The method as in claim 1 wherein said parathyroid hormone is administered parentally.

12. The method as in claim 1 wherein said parathyroid hormone is PTH(1-34) and said daily dose is 20 μg.

13. The method as in claim 1 wherein said treatment increases bone mass.

14. The method as in claim 1 wherein said treatment improves bone structure.

15. The method as in claim 1 wherein said treatment stabilizes one or more teeth.

16. The method as in claim 1 wherein said treatment reduces further bone loss at said tooth socket.

17. (canceled)

18. The method as in claim 1 wherein said parathyroid hormone is administered by local injection at or near said tooth socket.

19. A method to improve the prognosis of a dental implant procedure in a patient having inadequate alveolar bone mass, comprising administering human parathyroid hormone to said patient at a daily dose of 20 μg to 30 μg for a period of from about 3 months to about 6 months prior to said procedure.

20. A method to accelerate osseointegration of a dental implant in a patient following placement of an implant, comprising administration of human parathyroid hormone at a daily dose of 20 μg to 30 μg.
